(19) United States
(12) Patent Application Publication
(10) Pub. No.: US 2007/0141021 A1
(43) Pub. Date: Jun. 21, 2007

(54) Methylmaleimidyl polymer derivatives

(76) Inventors: Perry Rosen, North Caldwell, NJ (US); Kwang Nho, Walnut Creek, CA (US)

Correspondence Address: GIBBONS P.C., ONE GATEWAY CENTER, NEWARK, NJ, 07102, US

(21) Appl. No.: 11/638,997

(22) Filed: Dec. 13, 2006

Related U.S. Application Data
(60) Provisional application No. 60/751,473, filed on Dec. 19, 2005.

(51) Int. Cl. A61K 38/17; C07K 14/47; C07D 207/40

(52) U.S. Cl. 424/78.27; 530/350; 514/12; 525/54.1; 548/546

(57) ABSTRACT

Novel N-2-methylmaleirnide pegylating reagents for the site specific pegylation of therapeutically active proteins and methods for such pegylation using said reagents.

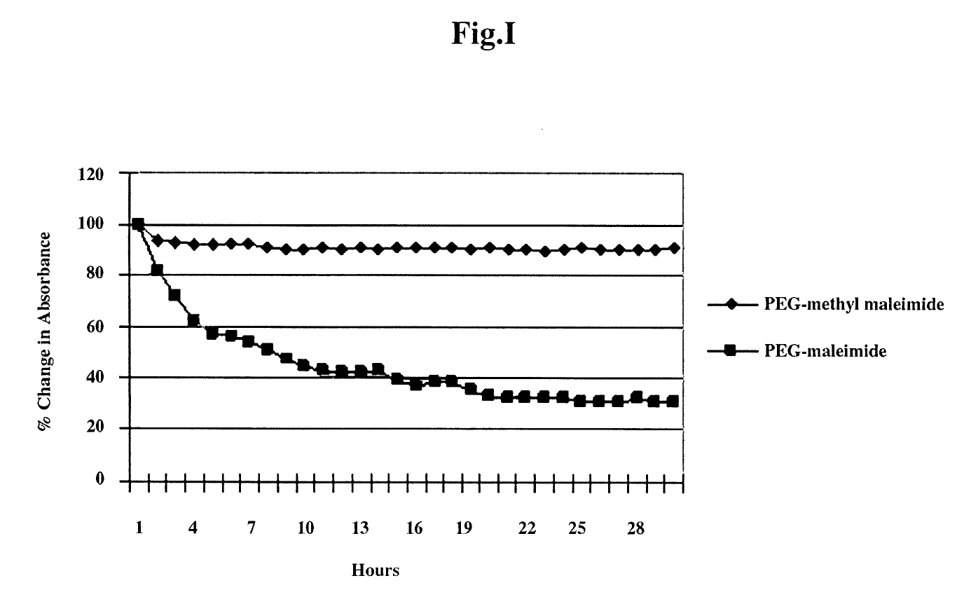

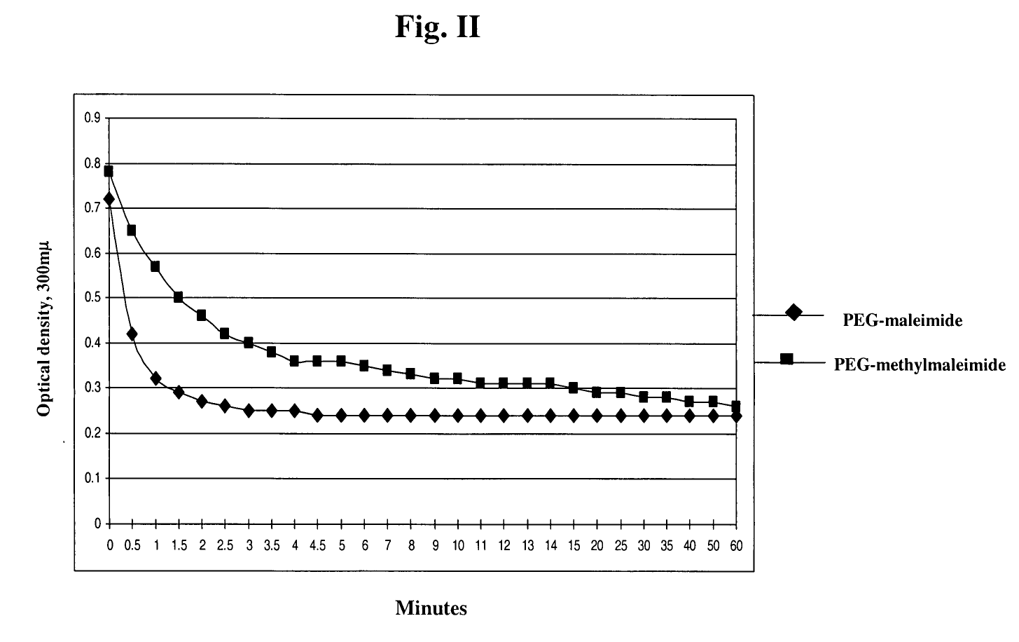

### CROSS REFERENCE TO RELATED APPLICATIONS

This Application claims priority of U.S. Provisional Ser. No. 60/751,473 filed Dec. 19, 2005, incorporated herein by reference.

### FIELD OF THE INVENTION

The field of this invention is pegylating therapeutic proteins and reagents for such pegylation.

### BACKGROUND

Therapeutic proteins which are generally administered by intravenous injection may be immunogenic, relatively water insoluble, and may have a short in vivo half-life. The pharmacokinetics of a particular protein will govern the efficacy and duration of effect of the drug. It has become of major importance to reduce the rate of clearance of the protein so that prolonged action can be achieved. This may be accomplished by avoiding or inhibiting glomerular filtration which can be effected both by the charge on the protein and its molecular size (Brenner et al. (1978) Am. J. Physiol. 234, F455). By increasing the molecular volume and by masking potential epitope sites, modification of a therapeutic polypeptide with a polymer such as polyethylene glycol (PEG) has been shown to be efficacious in reducing both the rate of clearance as well as the antigenicity of the protein. Reduced proteolysis, increased water solubility, reduced renal clearance, and steric hindrance to receptor-mediated clearance are a number of mechanisms by which the attachment of a PEG polymer to the backbone of a polypeptide may prove beneficial in enhancing the pharmacokinetic properties of the drug. Thus Davis et al. U.S. Pat. No. 4,129,337 discloses conjugating PEG to proteins such as enzymes and insulin to produce a less immunogenic product while retaining a substantial proportion of the biological activity.

PEG modification requires activation of the PEG polymer. This is accomplished by the introduction of an electrophilic center, thereby enabling the PEG reagent to be susceptible to nucleophilic attack, predominantly by the nucleophilic epsilon-amino group of a lysyl residue. Because of the number of surface lysines present in most proteins, the PEGylation process can result in random attachments leading to mixtures which are difficult to purify and which may not be desirable for pharmaceutical use.

There are a large variety of PEG reagents that have been developed for the modification of proteins. This involves the covalent attachment of a PEG molecule via the formation of a linking group between the PEG polymer and the protein (see for example Zalipsky, et al. and Harris et al. in: Poly (ethylene glycol) Chemistry: Biotechnical and Biomedical Applications; J. M. Harris ed. Plenum Press: New York, 1992, chap. 21 and 22). Some of these reagents are, to various degrees, unstable in the aqueous medium in which the PEGylation reaction occurs. The conjugation process often results in the loss of in vitro biological activity. This is due to several factors, foremost of which being a steric interaction with the proteins active sites. One method to help avoid these problems is to use a site-selective PEG reagent that will react with functional groups other than amines. The pegylation of a free cysteine moiety is the primary method by which the site-specific pegylation of a protein may be realized. A PEG-sulfhydryl reactive maleimide derivative may react with a thiol containing protein to yield exclusively the Michael addition compound. 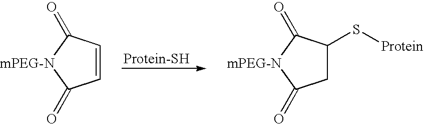
The selective derivatization of a cysteine moiety is made possible by the fact that a maleimide specific sulfhydryl reagent can form a covalent bond with a cysteine residue about 1000-fold faster than with a corresponding amine. The use of PEG maleimide reagents for the covalent attachment of poly (ethylene glycol) to various proteins has been reported for many years. (see: U.S. Pat. No. 4,810,638 to Albarella et al. Goodson et al. (1990) Bio/Technology 8, 343, Kogan (1992) Synthetic Comm. 22, 2417, U.S. Pat. No. 5,166,322 to Shaw et al. U.S. Pat. No. 5,206,344 to Katre et al. U.S. Pat. No. 5,766,897 to Braxton, and Tsutsumi et al. (2000) PANS 97, 8548). More recently, this technology has been applied to antibodies and antibody fragments in which specific cysteine residues are made accessible for PEG attachment (see A. P. Chapman (2002) Adv. Drug. Delivery Rev. 54, 531).

It is now recognized that the maleimide moiety is only temporarily stable towards hydrolytic cleavage and that decomposition to a non-reactive cis-maleamic acid derivative can occur during storage and in an aqueous reaction medium. (see: Wong, S. S., Chemistry of Protein Conjugation and Cross-Linking; CRC Press, 1993, 30; Roberts et al. (2002) Advanced Drug Delivery Reviews 54, 466; and U.S. Pat. No. 6,875,841 to Sakanoue et al.). 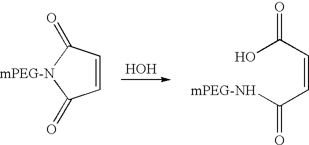
Furthermore, although the thioether linkage of the conjugation product is a very stable one and is not reversed under physiological conditions, slow hydrolytic cleavage of the succinimide ring can occur resulting in the formation of the open- ring succinamide acid derivatives. (see: Ishii et al. (1986) Biophys. J. 50, 75; Partis et al. (1983) J. Protein Chem. 2, 263; and U.S. patent publication Ser. No. 2006/0009590 to Kozlowski et al.) 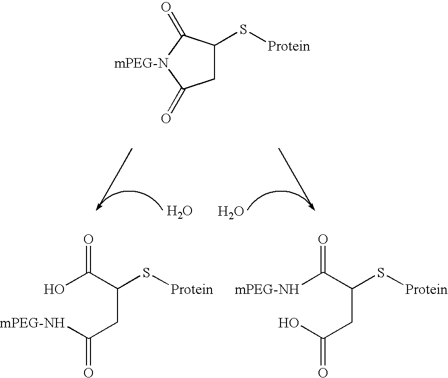
The importance of in vivo hydrolytic stability is exemplified by the case of the PEG maleimide modified αFab′ fragment, of an anti-TNF-α antibody (tumor necrosis factor (x) which resulted in the formation of a compound with a serum half-life of approximately 14 days. Its effectiveness as a treatment strategy in rheumatoid arthritis, was shown to be directly related to its extended plasma half-life (Choy et al. (2002) Rheumatology 41, 1133). Thus, the hydrolytic instability of the water soluble PEG maleimide reagent and its conjugation product demonstrates the need for the development of new sulfhydryl specific activated PEGs that are more resistant to hydrolytic cleavage and which can react with alacrity with biologically active molecules to give conjugated products which show an enhanced degree of in vivo stability.

### SUMMARY OF INVENTION

In accordance with this invention it has been discovered that when the methylmaleimidyl functional group of this formula: 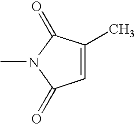
is used in place of the maleimidyl functional group of the formula 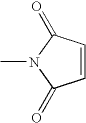
in pegylating reagents for forming pegylated protein conjugates , the reagents are stable against hydrolytic cleavage and eliminate the problems of the hydrolytic instability that occurs with pegylating reagents containing the unsubstituted maleimidyl functional group for reaction with the therapeutically active protein.

In forming the pegyled proteins from the pegylating reagents of this invention, the protein containing the sulfhydryl group is reacted with the methylmaleimidyl pegylating reagent to form a conjugated product via a conventional Michael addition. Previous work has shown that N-alkyl methylmaleimides will add sulhydryl groups (Miyadera et al. (1972) J. Med. Chem. 15, 534 and Earl et al. (1978) J. Heterocyclic Chem. 15, 1479).

The reaction is illustrated by the following reaction scheme:

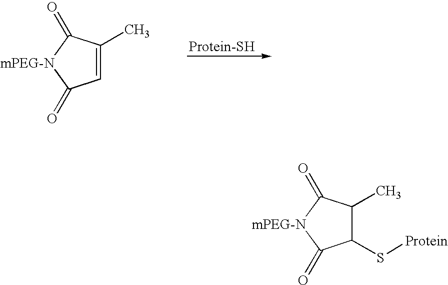

The Michael type addition reaction as illustrated in the above reaction scheme is generally carried out in an aqueous medium. By utilizing the methylmaleimide as the pegylating reagent, hydrolytic stability is provided to both the maleimide ring of the reagent and the succinimide ring of the protein polyoxyalkylene conjugate. Therefore through the use of the methylmaleimidyl functional group, rather than other functional groups such as the unsubstituted maleimidyl functional group, one produces pegylating reagents and pegylated protein conjugates which are more resistant to hydrolytic cleavage. Furthermore, one produces reactants that can react with alacrity with biologically active proteins containing a reactive sulfhydryl group to give pegylated protein conjugates which have an enhanced degree of in vivo stability.

The pegylating reagent of this invention are compounds which contain a polyoxyalkalene portion having a molecular weight from about 1,000 to 100,000 Daltons and which portion is covalently linked to a reactive terminal methylmaleimide functional group having the formula given above.

### BRIEF DESCRIPTION OF FIGURES

FIG. I describes the rates of disappearance of the UV absorption of the MPEG-methylmaleimide and mPEG-maleimide derivatives after dissolving each compound at room temperature in a phosphate buffer at pH 7.4. A half-life of approximately eight hours was determined for the maleimide compound whereas there was little change during a twenty four hour period for the methylmaleimide derivative.

FIG. II describes the rates of disappearance of the UV absorption of the mPEG-methylmaleimide and mPEG-maleimide derivatives respectively after dissolving each compound in a phosphate buffer at pH 6.5 followed by the addition of glutathione. As expected the rate of addition of the glutathione to the methylmaleimide compound is slower than that observed for the unsubstituted derivative.

### DETAILED DESCRIPTION

As seen from the foregoing, this invention is directed to novel reagents for polyoxylaklyating therapeutic proteins which contain a reactive sulfhydryl group. These new pegylating reagents are compounds that contain a polyoxyalkalene portion having a molecular weight of from about 1,000 to about 100,000 Daltons covalently linked to a reactive terminal methylmaleamide functional group having the formula; 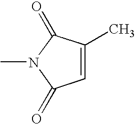
In accordance with this invention, the methylmaleamide functional group can be used as the reactive functional group in any of the conventional pegylating reagents which are used to produce polyoxyalkylene conjugates. By this invention one can obtain new and improved reagents to pegylate proteins containing a sulfhydryl functional group to produce polyoxyalkylene conjugates with therapeutic proteins. Pegylating reagents and their use in forming pegalyted conjugates with therapeutic proteins are well known. Note U.S. Pat. No. 5,298,643 Greenwald, , U.S. Pat. No. 5,643,575 Martinez et al.; U.S. Pat. No. 6,113,906 Greenwald et al.; U.S. Pat. No. 5,349,001 Greenwald et al.; U.S. Pat. No. 5,932,462 Harris et al.; U.S. Pat. No. 5,681,567 Martinez et al.; U.S. Pat. No. 5,919,455, Greenwald et al. and U.S. Patent Publication 2006/009590 Kozlowski et al. The pegylating reagents are compounds containing three parts. The parts of the pegylating reagent constitute the polyoxyalkene portion, the linking portion and the reactive functional portion. It is the reactive functional group which attaches the polyoxyalkene portion to the protein to form the conjugate. In these reagents the reactive functional group is at a terminal position and the polyoxyalkene portion is covalently linked to the terminal functional group by the linking portion. . Unsubstituted maleimidyl has been used as the reactive functional group in the pegylating reagents such as those shown in U.S. Patent Publication 2006/009590 Kozlowski et al.

These reagents contain a polyoxyalkylene portion with a molecular weight of from about 1,000 to about 100,000 Daltons which are covalently linked to the reactive functional group which in the case of the reagents of this invention is the reactive terminal methylmaleimidyl group. In accordance with the preferred embodiment of this invention, the polyoxoalkylene is poloxyethylewne. Generally it is preferred that the polyoxyalkyene portion have a molecular weight from about 5,000 to about 60,000 Daltons being preferred, with ranges of from about 10,000 to about 50,000 Daltons and from about 10,000 to about 30,000 being especially preferred. The polyoxyalkylene portion is generally attached to the functional group via a linking group. Any of the conventional linking groups which are utilized in such reagents can be utilized in the reagent of this invention. The linking group in the reagent of this invention can contain various sites in which the polyoxyalkylene groups which form the polyoxyalkylene portion is attached. The polyoxyalkylene portion can contain multiple polyoxyalkylene groups. The polyoxyalkylene groups are attached at different sites on the linking group which covalently links theses multiple polyoxyalkylene groups to the reactive terminal methylmaleimidyl functional group. Generally, it is preferred that the linking group contains 1 or 2 reactive sites in which the polyoxyalkylene groups are covalently attached. In addition, it is generally preferred that the pegylated portion contain from 1 to 2 polyoxyalkylene groups covalently linked through one or two linking group to the methylmaleimidyl function group. When the polyoxyalkylene portion consists of two polyoxyalkylene groups and one linking group each of these polyoxyalkylene groups are separately covalently attached at a different site on the linking group which linking group is covalently connected to the functional group. The formation of these pegylating reagents having a polyoxyalkylene portion consisting of two polyoxyalkylene groups, each being separately covalently attached to a different site on the linking group covalently attached to the functional group are described in various U.S. Patents such as those herein before mentioned particularly U.S. Pat. No. 5,919,455, Greenwald, et al. and U.S. Pat. No. 5,932,462 Harris et al.

In addition, the reagents of this invention can contain other reactive functional groups, in addition to the methylmaleimidyl functional group, to produce a poly-functional reagent. These other functional groups are reactive with the functional groups contained by the proteins. These other functional groups in the poly-functional reagents of this invention are linked by a second linking group placed at the end of a polyoxyalkylene group opposite the end where this polyoxyalkylene group is linked to the first functional methylmaleimidyl group. These other functional groups can be another methylmaleimidyl or any different functional group which is capable of reacting with a functional group on a protein. These poly-functional reagents can be used in forming a polyoxyalkylene conjugates by reacting with a plurality of functional groups at different sites on a given protein or by forming a polyoxyalkylene conjugate with two or more proteins. Generally, it is preferred that the reagent contain no more than two functional groups, with one being a methylmaleimidyl functional group which binds to the sulfhydryl group of the protein and the other being a conventional functional group for binding to another site on the protein or to another protein. Where poly functional reagents of this invention contain a plurality of functional groups, it is preferred that these other functional groups are also methylmaleimidyl. This will give better results in both reactivity and hydrolytic stability. In any case mono functional reagents are especially preferred.

Any of the conventional functional groups reactive with proteins can be used as the other functional group in the poly-functional reagents in conjunction with the methylmaleimidyl functional group contained in these reagents. These other functional group include, —OCH₃, —NH₂, —CHO, —COOH, —CH (OC₂H₅)₂, —OH, N-2-methylmaleimidyl, 1, 3-dioxolane, or any other functional group that is compatible with the chemical reactivity of the methylmaleimidyl group. These polyfunctional pegylating agents are said forth in U.S. Pat. No. 6,602,498; U.S. Pat. No. 6,541,543, U.S. Pat. No. 6,437,025 and U.S. Pat. No. 6,362,254.

The reagents of this invention can be reacted as described above with a protein containing a sulfhydryl group to form a pegylated protein conjugate. In accordance with this invention, these pegylating reagents are used to pegylate therapeutically active proteins by reacting the methymaleimidyl functional group of the pegylating reagent with the reactive sulfhydryl group of a therapeutic protein. In this manner a thio ether is formed from the pegylating reagent and the therapeutically active protein. By this reaction, the therapeutically active protein is conjugated with the polyoxyalkylene portion of the reagent. The reaction between the sulfhydyl group on the protein and a methylmaleimidyl functional group on the reagent is carried out utilizing conditions for carrying out Michael addition reactions. Any of the conditions conventional in carrying out Michael addition reactions can be used in this reaction. In this manner the polyoxyalklene protein conjugate is formed as a thioether which occurs when the sulfhydryl group is reacted with the terminal methylmaleimidyl functional group. The conjugate thus produced is hydrolytically stable. In addition by utilizing methylmaleimidyl functional group in the reagent, the Michael reaction can be carried out in an aqueous medium without the danger of hydrolytic cleavage.

Among the specific embodiments, the pegaylated reagents which can be utilized in accordance with this invention are compounds of the formula: 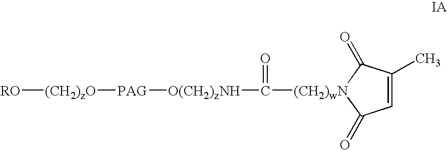
wherein R is hydrogen or lower alkyl, PAG is a divalent residue of polyalkylene glycol resulting from removal of the terminal hydroxy groups, having a molecular weight of from 1,000 to 100,000 Daltons, z is an integer of from 2 to 4, and w is an integer of from 1 to 8. 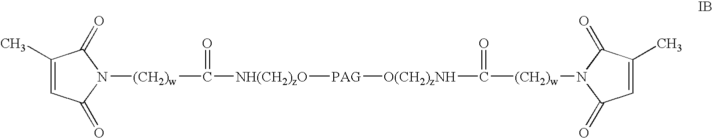
   wherein PAG is a divalent residue of polyalkylene glycol resulting from removal of the terminal hydroxy groups, having a molecular weight of from 1,000 to 100,000 Daltons, z is an integer of from 2 to 4, and w is an integer of from 1 to 8. 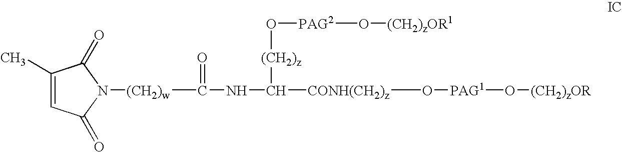
   wherein PAG¹ and PAG² are independently divalent residues of poly lower alkylene glycol resulting from removal of the two terminal hydroxy groups with the PAG¹ and PAG² residues having a combined molecular weight of from 1,000 to 100,000 Daltons, R and R¹ are individually lower alkyl or hydrogen, and z and w are as above. 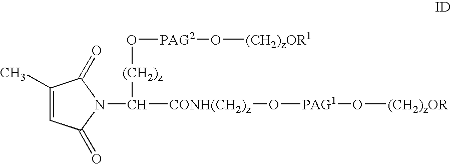
wherein PAG¹ and PAG² are independently divalent residues of poly lower alkylene glycol resulting from removal of the two terminal hydroxy groups with the PAG¹ and PAG² residues having a combined molecular weight of from 1,000 to 100,000 Daltons, R and R¹ are individually lower alkyl or hydrogen, and z and w are as above. 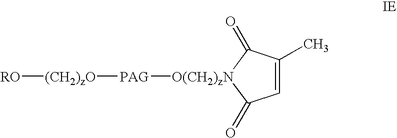
wherein R is hydrogen or lower alkyl, PAG is a divalent residue of polyalkylene glycol resulting from removal of the terminal hydroxy groups, having a molecular weight of from 1,000 to 100,000 Daltons, z is an integer of from 2 to 4. 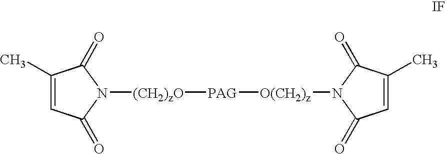
wherein PAG is a divalent residue of polyalkylene glycol resulting from removal of the terminal hydroxy groups, having a molecular weight of from 1,000 to 100,000 Daltons, and z is an integer of from 2 to 4.
   are useful for conjugation to therapeutically active proteins to produce PAG protein conjugates which retain a substantial portion of their therapeutic activity and are less immunogenic than the protein from which the conjugate is derived.

In accordance with this invention, the methods of preparation of both linker and linkerless N-PEG-2-methylmaleimide derivatives are provided.

The maleimide reagents of formula IA, IB, IC, ID, 1E, and IF can be conjugated to therapeutically active proteins and non-peptidyl derivatives to produce therapeutically active conjugates which retain a substantial portion of the biological activity of the compound from which they are derived. In addition, the reagents of this invention are more resistant to aqueous hydrolytic degradation as compared to the standard non-methylated derivatives. The maleimide reagents of this invention can be used for the site specific conjugation to a particular cysteine residue which is naturally occurring or which was engineered into a polypeptide backbone at a desired site. In this way, these maleimides produce the desired hydrolytically stable conjugates and avoid random attachment leading to mixtures that are difficult to purify and which may not be desirable for pharmaceutical use. This is extremely advantageous since not only are the purification procedures expensive and time consuming but they may cause the protein to be denatured and thus bring about an irreversible change in the proteins tertiary structure.

Exemplary therapeutic proteins and protein classes in which a sulfhydryl group may be present or introduced, can be conjugated in accordance with this invention and may be any of the conventional therapeutic proteins. Among the preferred proteins are included interferon-alpha, interferon-beta, consensus interferon, G-CSF, GM-CSF, EPO, hemoglobin, interleukins, colony stimulating factor, as well as immunoglobulins such as IgG, IgE, IgM, IgA, IgD, antibodies and fragments thereof.

The term polyalkylene glycol designates poly (lower alkylene) glycol radicals where the alkylene radical is a straight or branched chain radical containing from 2 to 7 carbon atoms. The term “lower alkylene” designates a straight or branched chain divalent alkylene radical containing from 2 to 7 carbon atoms such as polyethylene, polypropylene, poly n-butylene, and polyisobutylene as well as polyalkylene glycols formed from mixed alkylene glycols such as polymers containing a mixture of polyethylene and polypropylene radicals and polymers containing a mixture of polyisopropylene, polyethylene and polyisobutylene radicals. The branched chain alkylene glycol radicals provide the lower alkyl groups in the polymer chain of from 2 to 4 carbon atoms depending on the number of carbon atoms contained in the straight chain of the alkylene group so that the total number of carbons atoms of any alkylene moiety which makes up the polyalkylene glycol substituent is from 2 to 7. The term “lower alkyl” includes lower alkyl groups containing from 1 to 7 carbon atoms, preferably from 1 to 4 carbon atoms, such as methyl, ethyl, propyl, isopropyl, etc. with methyl being especially preferred.

In accordance with a preferred embodiment of this invention, PAG in the compounds in formulas IA, IB, IC, ID, 1IF is a polyethylene glycol residue formed by removal of the two terminal hydroxy groups. Further, in accordance with this invention, in the compounds of formula IA, IB, IC, ID, 1E, and IF, PAG may have a molecular weight of from about 10,000 to 50,000 most preferably from about 20,000 to about 40,000. In the compounds of formulas IC and ID it is generally preferred that the radicals PAG¹ PAG² have a combined molecular weight of from about 10,000 to 50,000 and most preferably from about 20,000 to 40,000. The compounds of formula IA, IB, IC, ID, 1E, and IF are used in forming polyalkyleneoxy protein conjugates with polypeptides bearing a free and accessible sulfhydryl group to produce a therapeutically effective conjugate that has the therapeutic properties of the native protein. These conjugates when compared to the compounds from which they are derived, are less susceptible to renal and receptor-mediated clearance, show decreased antigenicity, diminished in vivo proteolysis, and increased water solubility. All of these factors can help make the conjugate a very effective therapeutic agent.

The N-PAG-2-methylmaleimido derivatives of this invention can be prepared by several methods. A PAG amino derivative IX containing at least one PAG polymer backbone with an average molecular weight of from 1,000 to 100,000 is reacted with citraconic anhydride X to give a mixture of citraconic acid mono amides Xa and Xb. Ring closure to the maleimide derivative VIb may then be effected by heating the mono amide mixture for several hours at about 100EC with acetic anhydride and sodium acetate (Kogan, T. P. (1992) Synthetic Communications 22, 2417). This process is illustrated in the following general scheme:

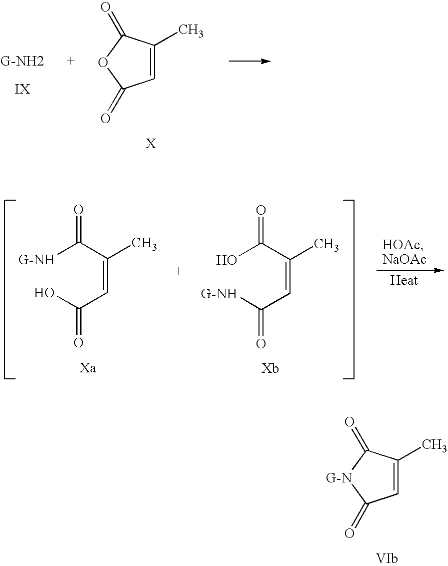

In accordance with another and preferred embodiment of preparing the above compounds, the intermediates Xa and Xb are reacted with pentafluorophenyl trifluoroacetate XI to give the active esters Xc and Xd which in a one step procedure cyclize to give the desired methylmaleimide derivative VIb. The carboxy groups of compounds Xa and Xb can also be activated by any of the other conventional methods such as with the use of N-hydroxysuccinimide.

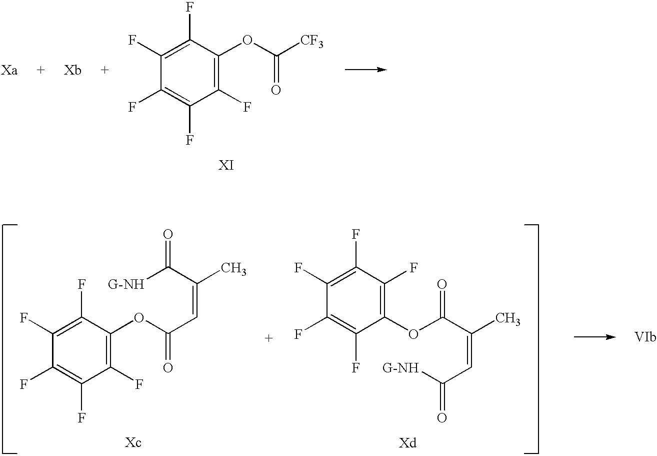

In the above reaction schemes, the compound of formula G-NH₂ is a composite of various multifunctional PAG polymer backbones having at least one terminus bonded to an amino group. The maleimide derivative VIb may represent the structures ID, IE, and IF.

In particular, the maleimide derivative XIIa which may represent compounds IA, IB, and IC, in which the maleimide group is attached to the PAG backbone by means of an amide linker, can be prepared by the reaction of a PAG amine IX and an active ester of a maleimido carboxylic acid XIII. The derivative XIII is formed by the reaction of compound XI with the mixture of Xe and Xf.

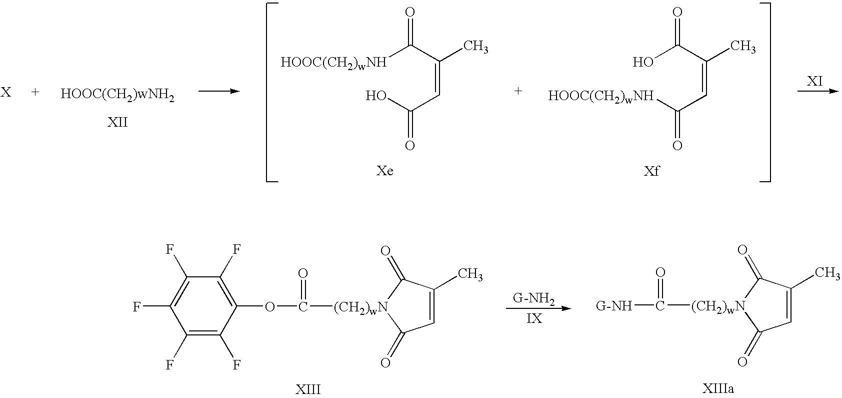

In the above reaction scheme, w and the compound of formula G-NH₂ are as above.

The maleimido reagents VIc of this invention may react with a sulfhydryl group of a protein to form a PEG-protein conjugate VIIa in accordance with the following scheme: 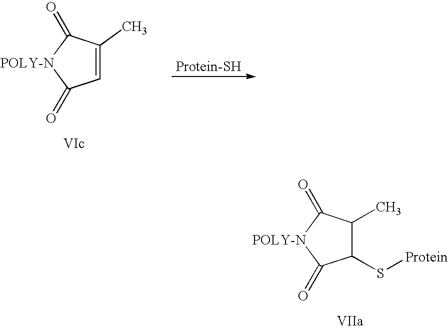
wherein PSH is a protein which can be covalently and selectively
attached to a methylmaleimidyl PEG derivative VIc to give VIla

In the above scheme, POLY-C₅H4NO₂ in the compound of formula VIc is a composite of the compounds of IA, IB, IC, ID, 1E, and IF showing the reactive methylmaleimido group. PSH is a protein containing a nucleophilic -SH group that is conjugated with the compounds of formula IA, IB, IC, ID, 1E, and IF to form a thioether. The thioether is formed at the carbon vicinal to the methyl group and is exemplified by compound VIIa.

Exemplary sulfhydryl coupling agents are the N-PAG-2-methylmaleimido groups which are specific to sulfhydryl groups, particularly at a pH below 7. The coupling of an appropriate cysteine residue with the 2-methylmaleimide group found in compounds IA, IB, IC, ID, 1E and IF, results in the formation of a thioether linkage as shown in structure VIIa. The selective derivatization of a sulfhydryl group of a cysteine amino acid is made possible by the reactivity of sulfhydryl specific reagents which can form covalent bonds with the cysteine residues at a rate which is many times faster than the reaction with a corresponding amine.

In reacting the compound of formula VIc with PSH, one can control this reaction so that the maleimides of formula IA, IB, IC, ID, 1E, and IF only react at a single accessible sulfhydryl site located on the protein backbone. This can be done by carrying out the reaction of the compound of formula VIc with PSH at a pH of from 5.5 to 7.5. In carrying out this reaction, various buffers which maintain the reaction media at a pH of from 5.5 to 7.5 can be used. The specific PEGylating reagents of this invention, are stable in aqueous medium and are not subject to limiting hydrolytic decomposition under the conditions of the Michael addition reaction.

The compounds of formula IA can be prepared as described by the following reaction scheme: 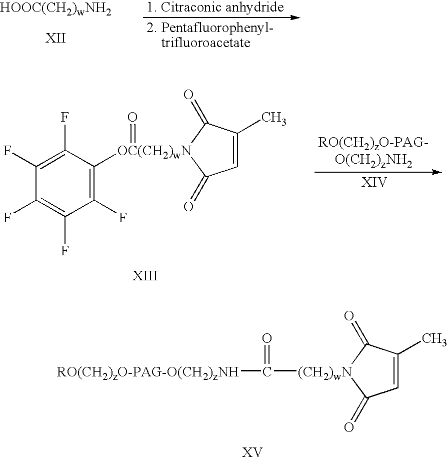
wherein R is hydrogen or lower alkyl, PAG is a divalent residue of polyalkylene glycol resulting from removal of the terminal hydroxy groups, having a molecular weight of from 1,000 to 100,000 Daltons, z is an integer of from 2 to 4, and w is an integer of from 1 to 8.

The compound of type XV, may be prepared from the amino acid XII by modification of a procedure described by Adamczyk et al. Org. Prep. Proced. Int. (1993) 25, 592. In carrying out this process, the amino acid XII is reacted with citraconic anhydride and pentafluorophenyl triflouroacetate in a one-pot procedure to give directly the methylmaleimidyl pentafluorophenyl ester derivative XHI. In the next step of the synthesis, the pentafluorophenyl ester compound XIII is reacted with the PAG amine compound the amide of formula XV is carried out by any conventional means of condensing an amine with an activated carboxylic acid group.

The compounds of formula IB can be prepared as described by the following reaction scheme: 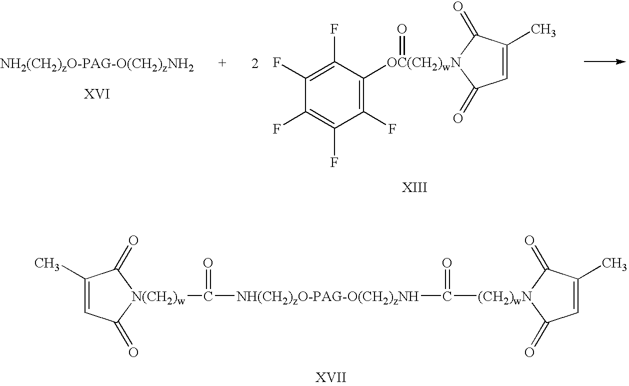
wherein, PAG is a divalent residue of polyalkylene glycol resulting
from removal of the terminal hydroxy groups, having a molecular
weight of from 1,000 to 100,000 Daltons, z and w are as above.

In carrying out this process the PAG diamine derivative XVI is reacted with two moles of the maleimido ester XIII to give the product XVII (IB).

The compounds of formula IC can be prepared as described by the following reaction scheme: 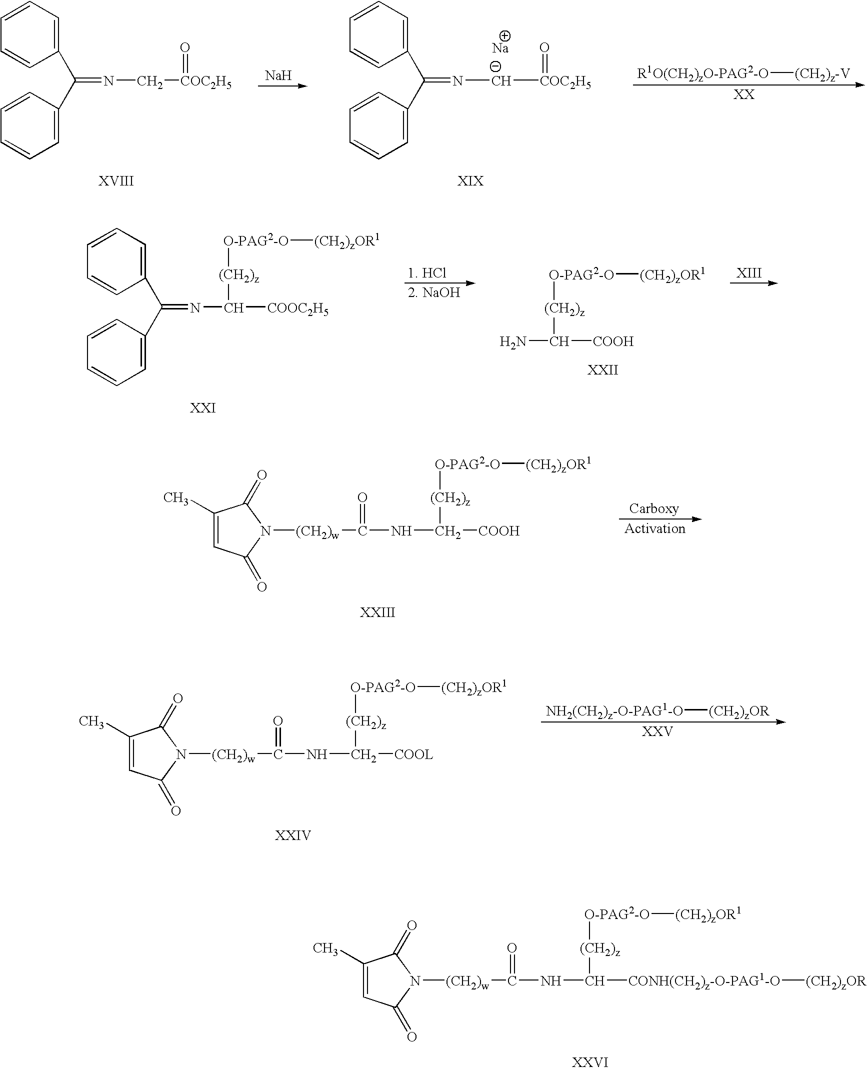
wherein PAG¹ and PAG² are independently divalent residues of poly lower alkylene glycol resulting from removal of the two terminal hydroxy groups with the PAG¹ and PAG² residues having a combined molecular weight of from 1,000 to 100,000 Daltons, V is tosyl, tresyl, bromine, or iodine, OL is an activating leaving group, R and R¹ are individually lower alkyl or hydrogen, and z and w are as above.

In this reaction sequence the compound of formula XX is condensed with the sodium salt XIX which is derived from the glycine Schiff base XVII by reaction with sodium hydride (for the alkylation of glycine derivatives see: a) O'Donnell et al. (1978) Tetrahedron Lett. 2641; b) Stork et al. (1976) J. Org. Chem. 41, 3491; and c) Bey et al. (1977) Tetrahedron Lett. 1455). Hydrolysis of the rsulting Schiff base XXI affords the alkylated glycine derivative XXII. The amino group of compound XXII may then be reacted selectively with compound XIII to give the maleimido amide XXIII. The acid group in formula XXIII is then converted to an active ester derivative XXIV. Any conventional method of activating a carboxylic acid by formation of an active ester such as an N-hydroxy succinimide ester, can be used to produce the compound of formula XXIV. In the next step of the synthesis, the compound of formula XXIV is then reacted with the PAG amine XXV to produce the compound of formula XXVI (IC). This reaction to form the amide of formula XXVI is carried out by any conventional means of condensing an amine with an activated carboxylic acid group.

The compounds of formula ID can be prepared as described by the following reaction scheme: 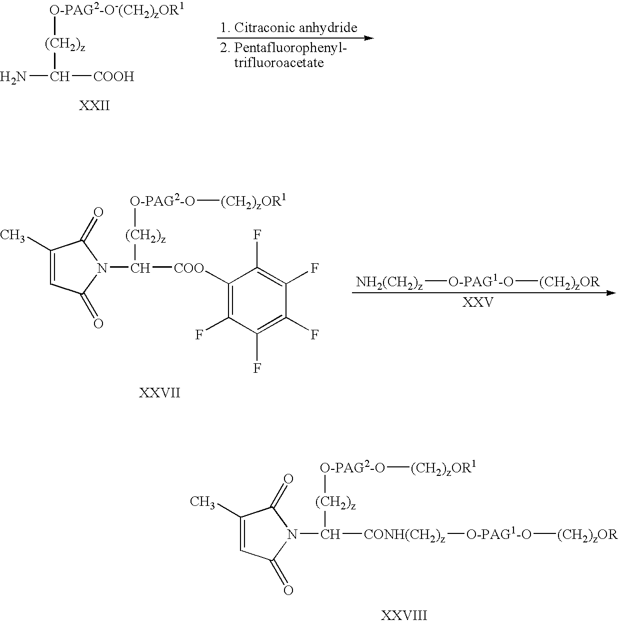
wherein PAG¹ and PAG² are independently divalent residues of poly lower alkylene glycol resulting from removal of the two terminal hydroxy groups with the PAG¹ and PAG residues having a combined molecular weight of from 1,000 to 100,000 Daltons, R and R¹ are individually lower alkyl or hydrogen, and z is as above.

The compound of type XXVIII may be prepared from the amino acid XXII by modification of a procedure described by Adamczyk et al. (1993) Org. Prep. Proced. Int. 25, 592. In carrying out this process, the amino acid XXII is reacted with citraconic anhydride and pentafluorophenyl triflouroacetate in a one-pot procedure to give directly the maleimidyl pentafluorophenyl ester-PAG² derivative XXVII. In the next step of the synthesis, the pentafluorophenyl ester of compound formula XXVII is reacted with the PAG¹ amine compound of formula XXV to produce the compound of formula XXVmI (ID). This reaction to form the amide of formula XXVIII is carried out by any conventional means of condensing an amine with an activated carboxylic acid group. The compounds of formula IE can be prepared as described by the following reaction scheme: 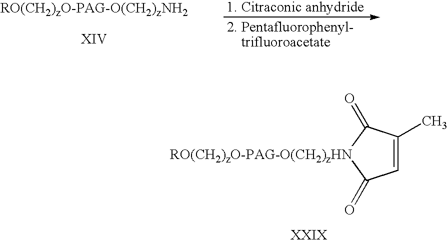
wherein R is hydrogen or lower alkyl, PAG is a divalent residue of polyalkylene glycol resulting from removal of the terminal hydroxy groups, having a molecular weight of from 1,000 to 100,000 Daltons, z is as above.

In carrying out this process, the PAG amine XIV is reacted with citraconic anhydride and pentafluorophenyl triflouroacetate in a one-pot procedure to give directly the maleimidyl derivative XXIX (IE).

The compounds of formula IF can be prepared as described by the following reaction scheme: 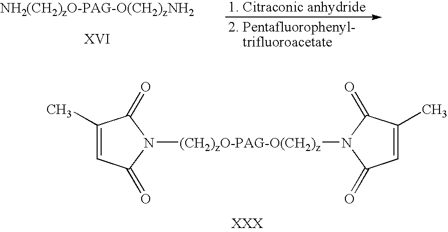
wherein PAG is a divalent residue of polyalkylene glycol resulting from removal of the terminal hydroxy groups, having a molecular weight of from 1,000 to 100,000 Daltons, and z is an integer of from 2 to 4.

In carrying out this process, the PAG diamine XVI is reacted with citraconic anhydride and pentafluorophenyl triflouroacetate in a one-pot procedure to give directly the N-PAG-2-methylmaleimide derivative XXX (IF).

### EXAMPLES

### Example 1

Preparation of

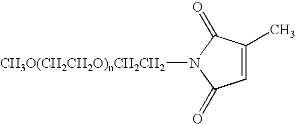

Citraconic anhydride 2 (290 mg, 2.5 mmol) was added to a solution of (o-methoxypoly-(oxyethylene)amine 1 (MW 20,000, 5 g, 0.25 mmol) in N,N-dimethylacetamide (20 ml) at 40EC for 2 h. The reaction was cooled, diluted with ether (250 ml) and refrigerated overnight. The citraconic acid mono amides 3 and 4 were filtered, washed with ether and dried under vacuum. The mixture of amides were then dissolved in a 4:1 mixture of methylene chloride and DMF (25 ml) to which was added diisopropylethylamine (2.5 mmol, 0.43 ml) and pentafluorophenyl trifluoroacetate 5 (2.5 mmol, 0.43 ml) and the solution stirred for 24 hrs at 50EC. The mixture was then slowly added with stirring to ether (200 ml) and the resulting mixture refrigerated for 12 hrs. The precipitate was filtered, washed with ether and dried under vacuum. The maleimide 6 was then dissolved in a minimum of methylene chloride and allowed to stand over activated charcoal. The mixture was filtered through celite, diluted with ether (250 ml) and cooled to OEC. The product was collected by filtration, washed with ether and vacuum dried to give compound 6. 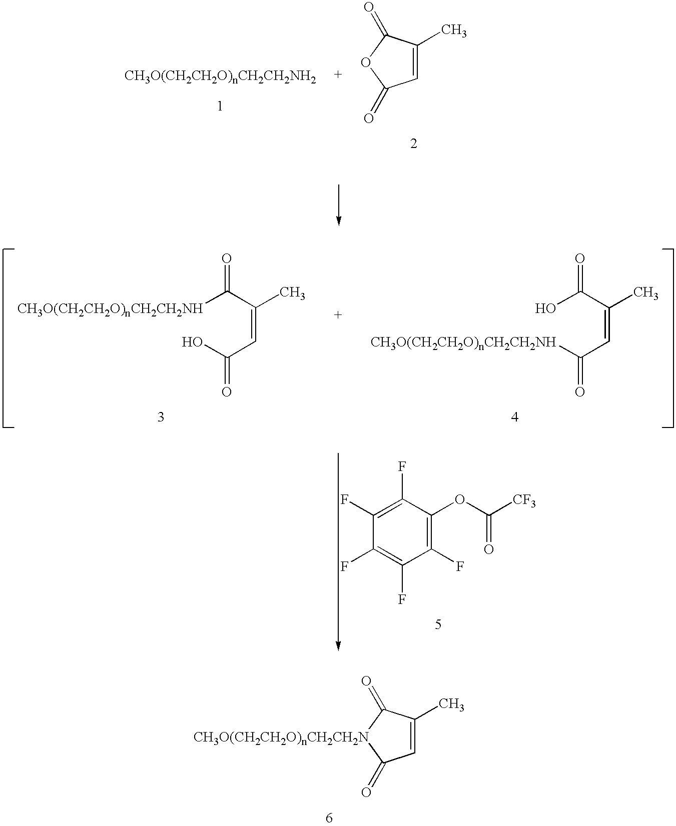
The integer n may be from about 20 to 2,300 but more preferably 20 to 1,000.

### Example 2

Preparation of

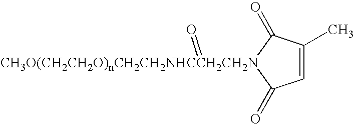

To a stirred solution of citraconic anhydride 2 (1.14 g, 10.2 mmol) in 5 ml of dry DMF was added 3-aminopropionic acid 7 (0.91 g, 10.2 mmol). After stirring for 8 hours under nitrogen, the reaction mixture which contained the citraconic acid mono amides 8 and 9 was cooled to OEC and diisopropylethylamine (4.4 ml, 25.5 mmol) dissolved in 6 ml of dry DMF was added followed by a solution of pentafluorophenyl trifluoroacetate 5 (7.2 g, 4.4 ml, 25.5 mmol) in 6 ml of the same solvent. The reaction was stirred at room temperature under nitrogen for 18 hours. Water (50 ml) was then added to the reaction mixture and extracted with methylene chloride. The methylene chloride solution was dried over magnesium sulfate and the solvent removed under reduced pressure to give the the petafluorphenyl-3-maleimido-propane-1-carboxylate 10. To 251 mg (0.75 mmol) of 10 dissolved in 30 ml of methylene chloride was added mPEG amine 11 (MW 20,000, 5 g, 0.25 mmol) and the solution stirred at room temperature for 12 hours. The product 12 was then precipitated by the slow addition of 250 ml of ether at OEC. The product was then washed with ether and dried under vacuum. 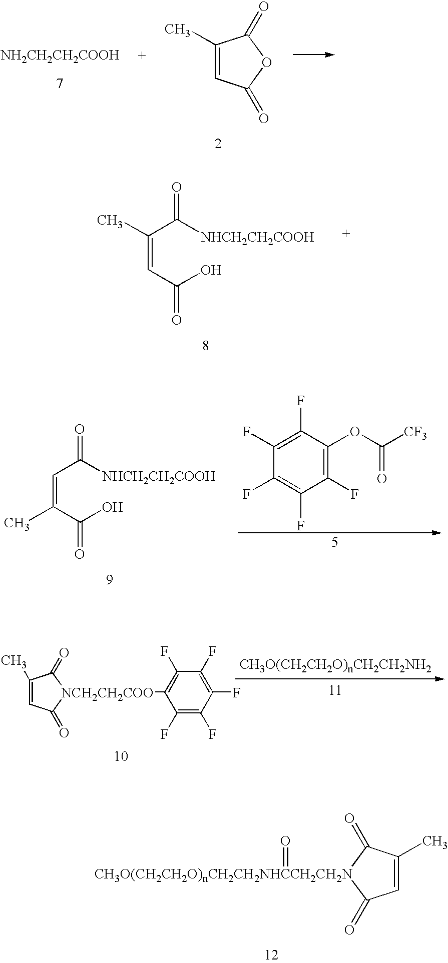
The integer n may be from about 20 to 2,300 but more preferably 20 to 1,000.

### Example 3

Preparation of

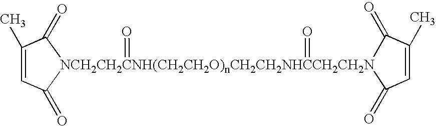

By following the same procedure as described in Example 2, the PEG dianine 13 is reacted with two moles of the 3-methylmaleimido ester 10, gives the product 14. 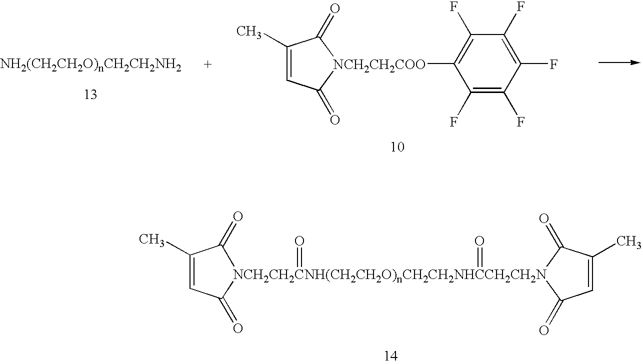
The integer n may be from about 20 to 2,300 but more preferably 20 to 1,000.

### Example 4

Preparation of

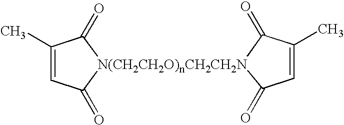

By following the same procedure as described in Example 1, the PEG diamine 13 is reacted with two moles of the citraconic anhydride 2 to give a mixture of mono amides which upon treatment with two moles of the pentafluorophenyl trifluoroacetate 5 gives the 2-methyl dimaleimido derivative 15. 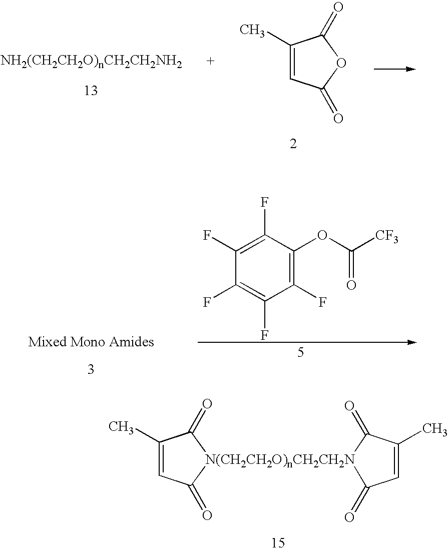
The integer n may be from about 20 to 2,300 but more preferably 20 to 1,000.

### Example 5

Preparation of

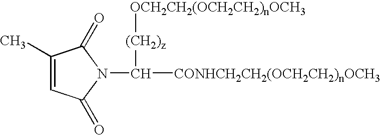

To a suspension of sodium hydride (24 mg, 1 mmol, 60% dispersion in mineral oil) in dry THF (10 ml), is added N-(diphenylmethylene)glycine ethyl ester 16 (267 mg, 1 mmol) dissolved in 10 ml of dry THF. The mixture is stirred for approximately 30 min at OEC after which time a light yellow solution is obtained which contains the sodium salt 17. To the solution is then added mPEG-I 18 (5 g, 0.25 mmol, 20,000 MW) dissolved in 50 ml of dry dioxane and 5 ml of hexamethylphosphoramide (PEG-I is prepared by a modification of the procedure described by Liu et al., (1995) Eur. Polym. J. 31, 819). The solution is warmed to 60EC for 24 hr and then treated with a small amount of an ice-cold aqueous solution of ammonium chloride. The mixture is then extracted with methylene chloride and dried over magnesium sulfate. The methylene chloride was removed under reduced pressure to give the Schiff base 19. Hydrolysis of compound 19 is accomplished by treatment with 5% HCl at room temperature for 6 hours. Extraction of the residue with methylene chloride afforded the amine salt which is then treated with 1N NaOH for twelve hours at room temperature. Extraction with methylene chloride, drying over magnesium sulfate and precipitation with ether afforded 3.8 g of the amino acid 20. Citraconic anhydride (116 mg, 1 mmol) is dissolved in N,N-dimethylacetamide (20 ml) to which is added 3.8 g (0.19 mmol) of the amino acid 20. The resulting solution is then heated at 60EC for 4 h. The solution is cooled to OEC and diisopropylethylamine (2.5 mmol, 0.43 ml) and pentafluorophenyl trifluoroacetate (2.5 mmol, 0.43 ml).is then added The reaction is then stirred at 40EC for 16 h, diluted with 25 ml of water and extracted with methylene chloride. The methylene chloride extracts are combined and dried over a mixture of MgSO₄ and activated charcoal. The mixture is then filtered through celite, diluted with ether (250 ml) and cooled to OEC to give the maleimido-ester 21. The product is collected by filtration, washed with ether and vacuum dried. Reaction of 21 and the MPEG amine 1 (1/1 mole ratio) in methylene chloride afforded the derivative 22. 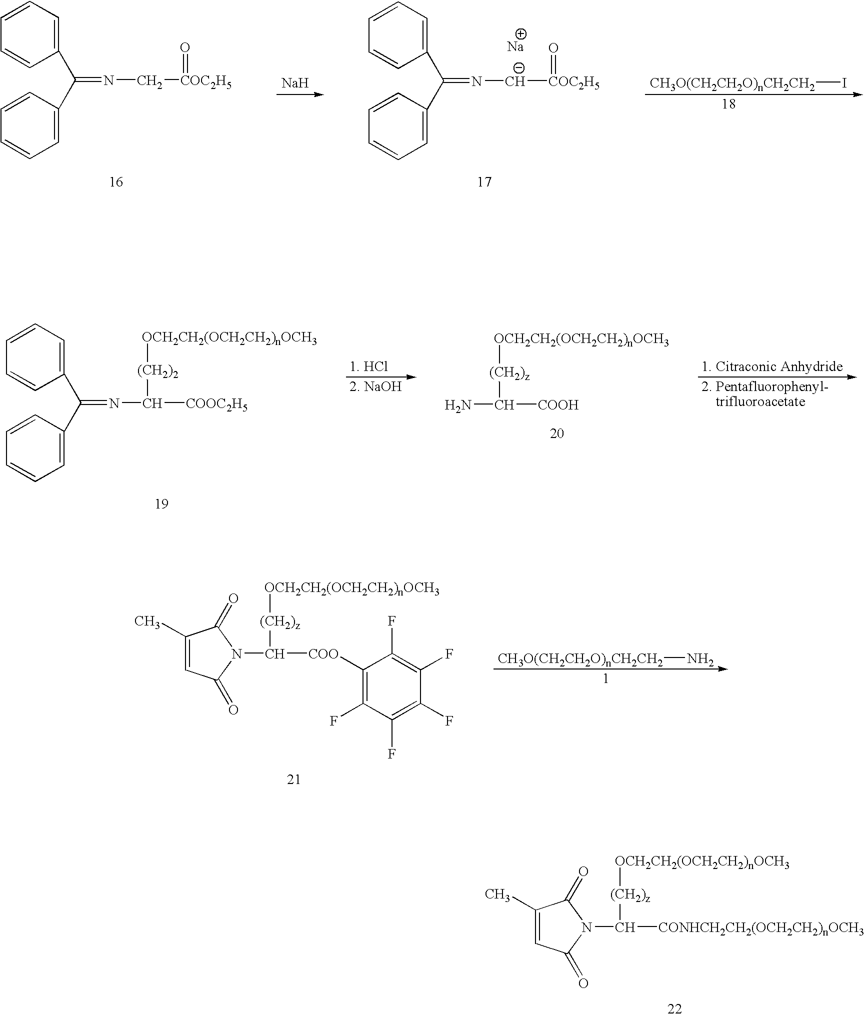
The integer n may be from about 20 to 2,300 but more preferably 20 to 1,000.

### Example 6

Preparation of

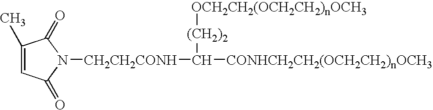

The reaction of the amino acid derivative 20 which is described in Example 5 is reacted with the maleimido ester 10 to give compound 23. The conditions for the condensation are as described in Example 2 for the reaction of the derivative 10 with a PEG amine. The carboxylc acid function in 23 is then converted to a pentafluorophenyl ester which when reacted with the PEG amine 1 affords the product 24 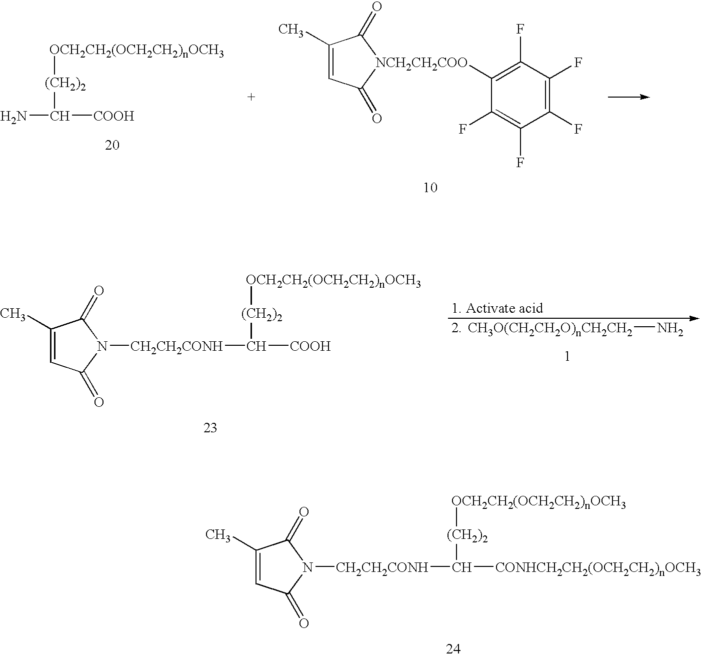
The integer n may be from about 20 to 2,300 but more preferably 20 to 1,000.

### Example 7

Preparation of

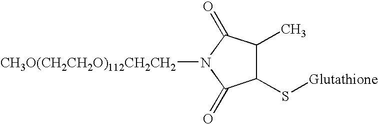

Glutathione (GSH) (10 mg, 3.58 mmoles) was added to a solution of compound 6 (1.5G, 0.3 mmoles) dissolved in 10 ml of 0.5 M phosphate buffer at pH 6.5. The reaction was run for six hours and the excess glutathione then removed by dialysis filtration. The aqueous solution was then extracted with methylene chloride, the extract dried (Na₂SO₄) and after precipitation with ether, the product was collected by filtration and dried under vacuum yield 25 (1.1 g). ¹H nmr (D₂O) indicates the predominant formation of the vicinally substituted derivative in which the methyl group of the thio-2-methylmaleimide addition product appears as a doublet centered at

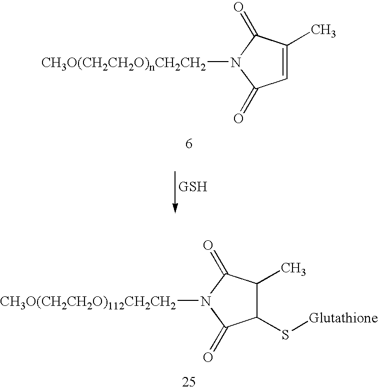

### Example 8

Hydrolytic Stability

mPEG-methylmaleimide (5,000) and mPEG-maleimide (5,000) were each dissolved in 20 mM phosphate buffer, pH 7.4 to make a 0.2 mM solution. The disappearance of the initial UV band at 300 nm was then monitored. The UV absorbance of the mPEG-maleimide declined by 50% after 10 hours and 70% after 30 hours. The UV absorbance of the mPEG-methylmaleimide declined by a negligible amount after 30 hours (FIG. I)

### Example 9

Kinetic Studies

mnPEG-methylmaleimide (5,000) and mPEG-maleimide (5,000) were prepared to 95% purity. Each compound was dissolved in 20 mM phosphate buffer at pH 6.5 and room temperature in order to form a 0.2 mM solution. Glutathione (61.4 mg, 2 mM) was added to each solution and the disappearance of the broad peak centered at 300 mp in the UV was used to follow the rate of reaction (FIG. II).

What is claimed:

1. A process for pegylating a protein having a sulfhydryl group to form a pegylated protein conjugate comprising reacting: 1) a compound containing a polyoxyalkylene portion with a molecular weight of from about 1,000 to 100,000 Daltons covalently linked to a reactive terminal methylmaleamidyl functional group having the formula: 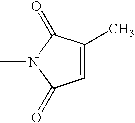
with 2) said protein having a sulfhydryl group on to form a pegylated protein conjugate by means of the thioether formed by said sulfhydryl group and said reactive terminal functional group.

2. The process of claim 1 wherein said polyoxyalkylene is polyoxyethylene.

3. The process of claim 2 wherein said compound contains a linking group which covalently links said polyoxyethylene portion to said terminal functional group.

4. The process of claim 3 wherein the compound contains one polyoxyethylene group covalently linked by means of said linking group to said terminal functional group.

5. The process of claim 3 wherein said linking group has a plurality of sites and said polyoxyethylene portion contains two polyoxyethylene groups, wherein each polyoxyethylene group is covalently attached at different sites on the linking group.

6. A pegylating reagent comprising a compound containing a polyoxyalkene portions having a molecular weight of from 1,000 to 100,000 Daltons covalently linked to a reactive terminal methylmaleamidyl functional group having the formula:

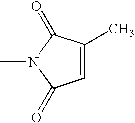

7. The reagent of claim 6 wherein the polyoxyalkylene is polyethylene.

8. The reagent of claim 7 wherein said compound contains a linking group which covalently links said polyoxyethylene portion to said terminal functional group

9. The reagent of claim 8 where the compound contains other functional groups in addition to said methylmaleamide functional group.

10. The reagent of claim 9 wherein said other functional groups are 3-methylmaleimidyl, —NH₂, —OR, —COOH, —CH(OR²)₂, —CHO, 1, 3-dioxolane, or N-hydroxysuccinimidyl, wherein R is hydrogen or lower alkyl and R² is lower alkyl.

11. The reagent of claim 10 wherein the compound contains one other functional group.

12. The reagent of claim 11 wherein the polyoxyethylene portion contains one polyoxyethylene group

13. The reagent of claim 11 wherein the polyoxyethylene portion has a molecular weight of from about 5,000 to about 60,000 Daltons.

13. The reagent of claim 12 wherein the polyoxyethylene portion has a molecular weight of from about 10,000 to about 30,000 Daltons.

14. The reagent of claim 10 wherein the polyethylene portion contains a single polyoxyethylene group linked by means of a linking group to each of said functional groups.

15. The reagent of claim 14 wherein the molecular weight of said polyoyetheylene goups is from about 5,000 to about 60,000 Daltons.

16. The reagent of claim 11 wherein said linking group has a plurality of sites and said polyoxyethylene portion contains two polyoxyethylene groups, wherein each polyoxyethylene group is covalently linked to said linking group at different sites on the linking group

17. The reagent of claim 16 wherein said other functional group is 3-methylmaleimidyl

18. The reagent of claim 17 wherein the collective molecular weight of said polyoxyethylene portion from about 5,000 to about 60,000 Daltons.

19. The reagents of claim 18 wherein the collective molecular weight of both of said protein portions is from about 10,000 to 30,000 Daltons.

20. The reagent of claim 8 wherein the compound contains said terminal methylmaleimidyl functional group as the only functional group.

21. The reagent of claim 20 wherein the polyoxyethylene portion contains a single polyethylene group linked by means of a linking group to said terminal functional group.

22. The reagent of claim 21 wherein the polyoxyethylene portion has a molecular weight of from about 5,000 to about 60,000 Daltons.

23. The reagent of claim 22 wherein the polyoxyethylene portion has a molecular weight of from about 10,000 to about 30,000 Daltons.

24. The reagent of claim 6, wherein said compound has the formula: 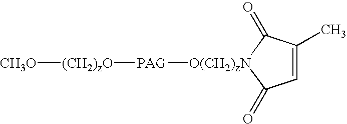
wherein PAG is a divalent residue of polyalkylene glycol resulting from removal of the terminal hydroxy groups, having a molecular weight of from 1,000 to 100,000 Daltons, z is an integer of from 2 to 4.

25. The reagent of claim 24 wherein said divalent residue is polyethylene glycol.

26. The reagent of claim 25 wherein the residue has a molecular weight of 5,000 to 50,000 Daltons.

27. The reagent of claim 6, wherein said compound has the formula: 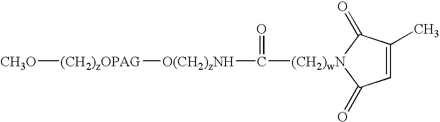
wherein PAG is a divalent residue of polyalkylene glycol resulting from removal of the terminal hydroxyl groups, having a molecular weight of from 1,000 to 100,000 Daltons, z is an integer of from 2 to 4, and w is an integer of from 1 to 8 and X is as above.

28. The reagent of claim 27 wherein said divalent residue is polyethylene glycol.

29. The reagent of claim 28 wherein the residue has a molecular weight of 5,000 to 50,000 Daltons.

30. The reagent of claim 6 wherein said compound has the formula: 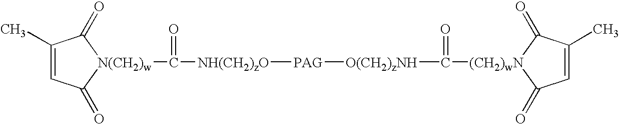
wherein PAG is a divalent residue of polyalkylene glycol resulting from removal of the terminal hydroxy groups, having a molecular weight of from 1,000 to 100,000 Daltons, z is an integer of from 2 to 4, and w is an integer of from 1 to 8.

31. The reagent of claim 30 wherein said divalent residue is polyethylene glycol.

32. The reagent of claim 31 wherein the residue has a molecular weight of 5,000 to 50,000 Daltons.

33. The reagent of claim 6, wherein said compound has the formula: 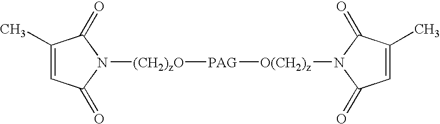
wherein PAG is a divalent residue of polyalkylene glycol resulting from removal of the terminal hydroxy groups, having a molecular weight of from 1,000 to 100,000 Daltons, and z is an integer of from 2 to 4.

34. The reagent of claim 33 wherein said divalent residue is polyethylene glycol.

35. The reagent of claim 34 wherein the residue has a molecular weight of 5,000 to 50,000 Daltons.

36. The reagent of claim 6 wherein said compound has the formula: 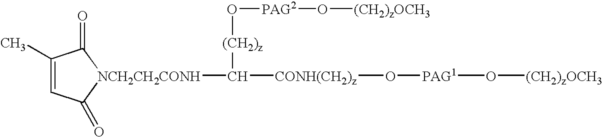
wherein PAG¹ and PAG² are independently divalent residues of poly lower alkylene glycol resulting from removal of the two terminal hydroxy groups with the PAG¹ and PAG² residues having a combined molecular weight of from 1,000 to 100,000 Daltons, m is an integer of from 0 to 1, and z is an integer of from 2 to 4.

37. The reagent of claim 6, wherein said compound has the formula: 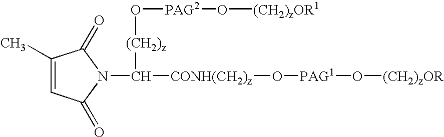
wherein R and R¹ are individually lower alkyl or hydrogen, PAG¹ and PAG and z are as above.

38. The reagent of claim 37 where PAG¹ and PAG² are each polyethylene glycol.

39. The reagent of claim 38 wherein PAG¹ and PAG² have a combined molecular weight of from 5,000 to 50,000 Daltons.
